(19) United States
(12) Patent Application Publication
(10) Pub. No.: US 2026/0361545 A1
(43) Pub. Date: Nov. 12, 2026

(54) TRUSTED EXECUTION ENVIRONMENT SYSTEM AND METHOD FOR EPHEMERAL ROOT OF TRUST USING CARDIAC LIVENESS AND QUANTUM ENTROPY FUSION

(71) Applicant: MICHAEL LEE CLARKE, Mountain View, CA (US)

(72) Inventor: MICHAEL LEE CLARKE, Mountain View, CA (US)

(21) Appl. No.: 19/722,855

(22) Filed: Jun. 28, 2026

Related U.S. Application Data
(60) Provisional application No. 63/919,438, filed on Nov. 17, 2025.

(51) Int. Cl. H04L 9/32; A61B 5/117; A61B 5/346; H04L 9/08

(52) U.S. Cl. CPC H04L 9/3231; A61B 5/117; A61B 5/346; H04L 9/0869

(57) ABSTRACT

A self-contained, host-agnostic biometric-quantum cryptographic system provides a portable, ephemeral root of trust within a physical Trusted Execution Environment (TEE) boundary. An ingestion interface continuously captures a cardiac electrical waveform to extract a dynamic biological feature vector. A secure chip enclave determines liveness by monitoring a bias voltage associated with the waveform, such that detachment or signal discontinuity produces a detectable voltage change, while an integrated photonic quantum random number generator simultaneously provides a continuous true quantum entropy stream. A hardware fusion engine deterministically combines the feature vector and entropy stream via a key derivation function to generate an ephemeral quantum cardiac key token. A hardwired analog zeroization circuit, independent of firmware, flushes the token from memory within microseconds of a detected liveness failure or detachment. An outbound transport bridge cryptographically wraps the token within a session key prior to transmission, authenticating permissions across external networks without exposing the token.

### CROSS-REFERENCE TO RELATED APPLICATIONS

The present application claims the benefit of priority to U.S. Provisional Patent Application No. 63/919,438, filed Nov. 17, 2025, the entire contents of which are hereby incorporated by reference in their entirety.

### BACKGROUND OF THE INVENTION

### 1. Field of the Invention

The present disclosure relates generally to hardware-level cryptographic architectures and self-contained, host-agnostic identity authentication. More specifically, it relates to a portable hardware token that establishes a dynamic, biometric-quantum root of trust without reliance on static factory-fused silicon keys or externally-hosted entropy sources.

### 2. Description of the Related Art

Traditional hardware security modules, smart cards, and trusted execution environments rely on static cryptographic keys fused into silicon gates during manufacturing, often referred to as physically unclonable functions (PUFs) or burned-in master keys. These static roots of trust are vulnerable to physical-layer exploits, including side-channel analysis, differential power analysis, and focused ion beam (FIB) probing. If a static root key is extracted, compromised, or modeled by a hostile actor, the integrity of the downstream security infrastructure relying on that key is permanently compromised.

Existing solutions attempting to introduce biometric variability often utilize software-level verification matchers that operate outside the hardware's primary cryptographic generation pipeline. These configurations may expose raw biological data to interception or manipulation during standard operating system execution cycles. Similarly, network-dependent security tokens that rely on cloud-based or remotely-hosted Quantum Random Number Generators (QRNGs) introduce communication latency, additional attack surfaces over the communication link, and a dependency on continued network connectivity for entropy generation.

Accordingly, a need exists for a self-contained, portable token that fuses continuous biological liveness signals with true quantum entropy entirely on-board the hardware device, establishing a non-static, ephemeral identity profile that does not depend on a persistently stored cryptographic secret.

### SUMMARY OF THE INVENTION

The present disclosure is directed to a self-contained, host-agnostic, biometric-quantum cryptographic system that establishes a portable, ephemeral root of trust across both physical and digital networks. Rather than relying on static, factory-fused silicon keys prone to physical tampering, the system introduces a highly integrated, multi-domain entropy fusion mechanism that improves the underlying security architecture of the hardware device itself, enabling a non-static, tamper-resistant cryptographic state that conventional static-key hardware cannot achieve.

In accordance with one embodiment of the present disclosure, the system comprises an ingestion interface that continuously captures an active, time-varying cardiac electrical waveform from a living user to extract a dynamic biological feature vector. This biological vector is fed directly into a portable hardware token defined by a physical Trusted Execution Environment (TEE) cryptographic hardware boundary. This physical boundary houses and isolates a secure chip enclave executing a continuous liveness monitoring subsystem, and a photonic quantum random number generator die comprising a gallium arsenide (GaAs) zincblende-structured emitter, a silicon dioxide (SiO₂) waveguide, and a single-photon avalanche diode (SPAD) detector operative at ambient room temperature, for continuous generation of true quantum entropy from quantum-mechanical photon detection events. In a preferred embodiment, this photonic die is miniaturized using a thin-film lithium niobate (TFLN) integration platform to enable its incorporation within a compact wallet-card form factor while remaining fully enclosed within the TEE boundary. The boundary further houses a hardware-implemented cryptographic fusion engine. The liveness monitoring subsystem determines liveness by continuously monitoring a bias voltage associated with the live cardiac signal, such that a physical detachment or signal discontinuity is reflected as a detectable change in that bias voltage, rather than as an algorithmic inference alone.

In another aspect of the present disclosure, during an initial enrollment phase, the secure chip enclave extracts baseline heart rate variability metrics from the user and processes them through a hardware-implemented fuzzy extractor to synthesize a stable, deterministic biometric root key, together with helper data retained in non-volatile memory and structurally insufficient, on its own, to reconstruct the user's underlying biometric signal. This biometric root key is injected into a secure, non-volatile register isolated within the TEE hardware boundary. No debug interface, test-mode access, or firmware-mediated pathway exists by which this register can be read or exported beyond the TEE boundary; any attempted access through such a pathway is structurally blocked by the TEE boundary and the zeroization mechanisms described below. During subsequent use, variance in the live cardiac signal arising from normal physiological conditions is reconciled against the stored helper data, allowing the device to recover a match against the enrolled root key without re-exposing the user's raw biological signal.

In a further aspect of the present disclosure, the system maintains a transient, zero-state security profile for its derived cryptographic output. Within the TEE hardware boundary, the cryptographic fusion engine deterministically combines the live biological feature vector, validated against the enrolled root key, with the quantum entropy stream, using at least one key derivation function (KDF) primitive. This multi-domain fusion generates an ephemeral quantum cardiac cryptographic key (KQCK) token, distinct from and derived independently of the permanent biometric root key. A hardwired analog zeroization circuit, implemented independently of any firmware or software execution, directly monitors the bias voltage of the liveness monitoring subsystem; upon a detected liveness failure or physical detachment, the circuit collapses that bias voltage to drive a depletion-mode switch into a conducting state, shorting the fusion engine's volatile registers to a dedicated ground plane and physically depleting the ephemeral KQCK token within microseconds. As with the biometric root key, no debug, test-mode, or firmware pathway exists by which the ephemeral KQCK token may be accessed or exported other than through the transport mechanism described below.

In a still further aspect of the present disclosure, distinct from the key-generation and enrollment aspects described above, a method is disclosed for projecting the ephemeral KQCK token's authentication state outward to external systems, while the permanent biometric root key remains confined within the originating TEE boundary. An outbound host transport bridge operates in at least two modes: in a first mode, the bridge verifies permissions directly against distributed networks or host environments using the ephemeral token as a transient credential; in a second mode, intended for use with a host device containing a separate, third-party secure enclave, the bridge cryptographically wraps the ephemeral KQCK token within a session key or secure channel protocol prior to transmission via a near-field or short-range wireless interface, such that the third-party enclave receives only the wrapped token or a derived proof, and the unwrapped token is at no point exposed in the clear during transit. This enables the host device's existing security infrastructure to be augmented with a real-time biometric-quantum-derived credential, while the originating device's entropy generation, fusion process, and permanent biometric root key remain on-board, and the ephemeral token itself remains subject to the zeroization circuit described above for the duration of its validity.

### BRIEF DESCRIPTION OF THE DRAWINGS

FIG. 1 illustrates an exploded view of the QCK showing the multi-layer physical, material, and atomic construction of the portable hardware token in a wallet card form factor, and a fixed adapter module (112) comprising the same internal architecture in an alternative embodiment for integration with legacy access control or timekeeping devices.

FIG. 2 illustrates a high-level block diagram of the 5-Layer Architecture of the Quantum Cardiac Key (QCK) system, depicting the flow from the Human Liveness Layer and Quantum Entropy Layer, through the Fusion Engine, Secure Chip Foundation, and TFLN Miniaturization, to the Secure Key Output, together with a hardwired zeroization circuit (216) and a hardware fuzzy extractor (217) housed within the Secure Chip Foundation.

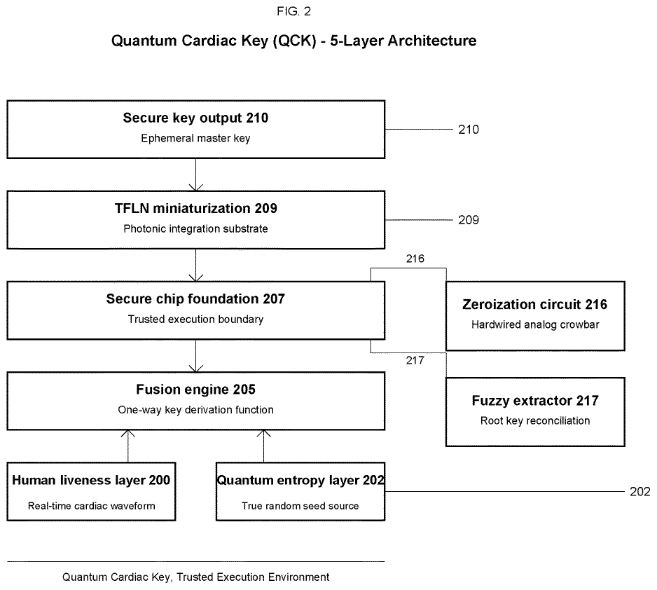

FIG. 3 illustrates a block diagram of a method for projecting an ephemeral cryptographic key token's authentication state to an external system, comprising a host transport bridge that cryptographically wraps the token prior to transmission via an interface and transport layer, and at least two operational modes by which the wrapped token, or a proof derived therefrom, authenticates user permissions to a distributed network, host environment, or third-party secure enclave, independent of the physical hardware token's internal architecture.

### DETAILED DESCRIPTION OF THE INVENTION

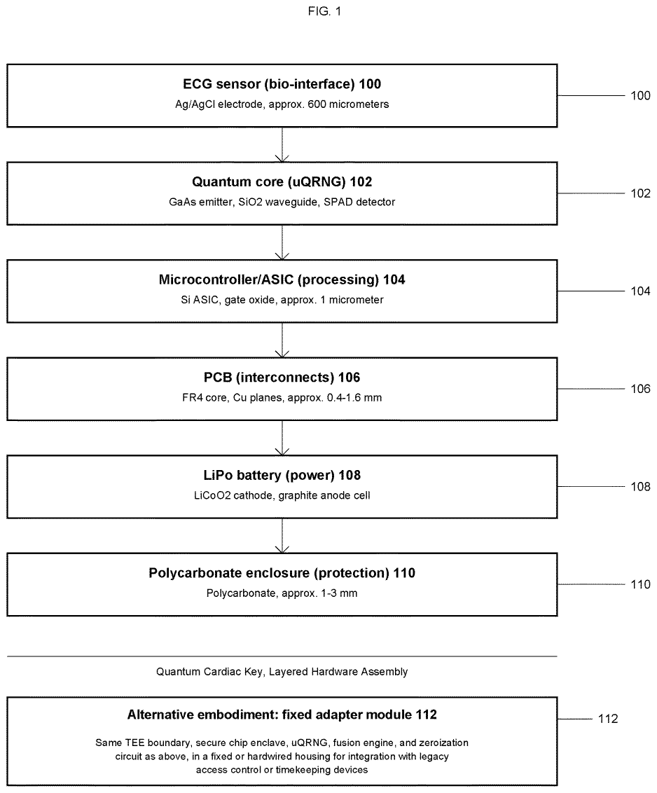

In one embodiment, the Quantum Cardiac Key (QCK) is implemented as a portable hardware token in the form of a tappable wallet card, as shown in FIG. 1. The QCK comprises a multi-layer architecture as follows (top-down layers):
ECG Sensor (Bio-Interface 100): Outer gel layer of amorphous H₂O/KCl (O—H covalent bonds, ~600 μm) for skin contact; AgCl ionic rock-salt layer (alternating planes); Ag substrate with FCC close-packed (12-coordinate atoms) structure. This ingestion interface uses at least two spatially separated, electrically isolated conductive contact nodes positioned along the exterior perimeter or opposite faces to capture a time-varying cardiac electrical waveform (ECG) from a living user (200) across a differential path.
Quantum Core (with μQRNG 102): Emitter: GaAs zincblende (Ga—As tetrahedral); Waveguide: SiO₂ tetrahedral (Si—O—Si amorphous); Detector: single-photon avalanche diode (SPAD), operative at ambient room temperature, for continuous true quantum entropy generation (202). In the preferred embodiment, the photonic micro-die μQRNG is miniaturized using a Thin Film Lithium Niobate (TFLN) platform (209) for integration into the compact wallet card form factor, enabling on-chip quantum entropy within the TEE boundary.
Microcontroller/ASIC (Processing 104): Si wafer (diamond cubic, tetrahedral bonds, ~1 μm); Doped channels (P/B substitutional, ~10¹⁶/cm³); Gate oxide (SiO₂ monolayer, 1-2 nm covalent); Metal traces (Cu FCC with grain boundaries). The processing layer derives HRV parameters from the raw ECG signal as needed for the feature vector.
PCB (Interconnects 106): Top Cu (FCC foil, 18-35 μm etched); Prepreg (Epoxy C—O amorphous crosslinks, ~100 μm); Core (FR4 SiO₂ fibers in epoxy, 0.4-1.6 mm); Inner/outer Cu laminated FCC planes.
LiPo Battery (Power 108): Anode collector (Al FCC foil); Anode (Graphite sp² hexagonal layers with Li intercalation); Separator (Polyethylene C—H amorphous, ~25 μm); Cathode (LiCoO₂ R-3 m octahedral, alternating planes); Cathode collector (Al FCC foil).
Polycarbonate Enclosure (Protection 110): Surface (amorphous helical C—C—O chains, van der Waals); Bulk (semi-crystalline phenyl π-stacks, ~1-3 mm); Filler (TiO₂ rutile octahedral, Si—O—C coupled).

The system operates with a Trusted Execution Environment (TEE) boundary enclosing the quantum core, fusion engine (205), and liveness monitoring. The ingestion interface feeds the dynamic cardiac-ionic ECG feature vector into the fusion engine, which combines it with the μQRNG entropy stream using an HKDF-based KDF. A hardwired zeroization pathway linked to liveness failure instantly erases the ephemeral KQCK token.
Alternative Embodiment (QCKa Adapter/Legacy Form Factor): In another embodiment, the QCK core stack is implemented in a non-card adapter module designed for fixed or fitted integration with legacy systems (e.g., time clocks, building access nodes).

### The Hardware-Level Zeroization Interrupt Pathway

The hardware-level zeroization interrupt pathway is implemented as a hardwired, analog crowbar circuit configured to bypass all system software execution cycles. The pathway directly monitors the bias voltage maintained by the continuous liveness monitoring subsystem (207).

Upon a detected liveness failure or physical card detachment, the localized bias voltage collapses, instantly driving a depletion-mode field-effect transistor (FET) switch into a conducting state. This conduction shorts the volatile memory registers of the cryptographic fusion engine (205) directly to a dedicated low-impedance ground plane. This hardware-driven charge dump forces an instantaneous physical depletion of the binary storage capacitors, executing a complete and irreversible destruction of the ephemeral KQCK token (210) within less than 1.2 μs of the detected failure.

### The Onboarding User Root Key Enrollment Protocol

During an initial configuration or enrollment phase of the device, an onboarding root key capture protocol is executed within a physically trusted environment to establish the device's permanent, localized cryptographic anchor. The ingestion interface (100) captures a baseline time-varying cardiac electrical waveform from the user, and the secure chip enclave (207) extracts baseline heart rate variability (HRV) metrics therefrom.

The enclave processes these baseline HRV metrics through a hardware-implemented fuzzy extractor primitive containing a cryptographic key generation block and an error-correcting helper data generator. The primitive maps the core statistical autonomic nervous system signatures to synthesize a stable, deterministic biometric root key, while generating helper data, stored in non-volatile memory, that is structurally insufficient on its own to reconstruct the user's underlying biometric signal. This biometric root key is injected directly into a secure, non-volatile register isolated within the physical TEE cryptographic hardware boundary to serve as the device's permanent, localized cryptographic anchor.

The biometric root key is structurally restricted from export outside the physical TEE cryptographic hardware boundary. Instead, during subsequent real-time authentication cycles, natural biological variance in active heart rate is reconciled by processing the live feature vector against the helper dataset within the fuzzy extractor's recovery block. Target external resources leverage downstream verification hashes derived from this registered reference anchor, allowing the token to cryptographically prove authenticity without exposing the user's raw underlying biological signals or local entropy parameters to an outside network.

### Network Paradigm and Outbound Execution

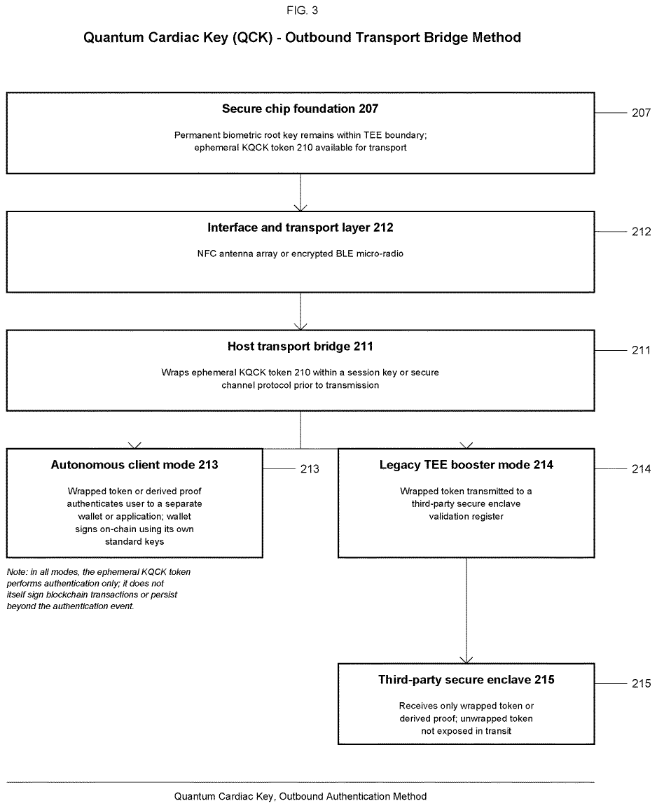

Referring now to FIG. 3, the portable hardware token communicates externally through an interface and transport layer (212), using a credit-card size ID-1 form factor equipped with an integrated near-field communication (NFC) antenna array or an encrypted Bluetooth Low Energy (BLE) micro-radio.

Using this interface, the token links via a host transport bridge (211) to project the ephemeral KQCK token's (210) authentication state outward to external resources, such as enterprise gates, distributed applications, or cloud servers. The system supports on-demand operation using at least two operational modes:
Autonomous Client Mode (213): The token uses its host bridge (211) to authenticate the user against distributed networks or host environments, using the ephemeral KQCK token (210), or a proof derived therefrom, as a transient credential that acts as a real-time biometric-quantum authentication factor to log into remote nodes or to authenticate the user to a separate wallet application or signing service. That wallet application retains and applies its own independent cryptographic signing keys (e.g., secp256k1) to authorize and broadcast any associated on-chain transaction. The ephemeral KQCK token itself does not sign transactions directly and is not retained by the wallet application beyond the authentication event, and at no point is the permanent biometric root key or any static credential transmitted.
Legacy TEE Cryptographic Booster Mode (214): When paired with a host device containing a separate, third-party silicon enclave (215) (e.g., a commercial mobile device or server chip), the portable token cryptographically wraps the ephemeral KQCK token (210) within a session key or secure channel protocol prior to transmission, such that the third-party enclave's (215) validation registers receive only the wrapped token or a derived proof, and the unwrapped token is at no point exposed in the clear during transit. This operation upgrades the host device's static legacy silicon to a dynamic, biometric-quantum-derived security state, while the primary entropy generation, fusion process, and permanent biometric root key remain entirely on-board and inaccessible outside the originating physical TEE boundary (207).

1. A self-contained, host-agnostic biometric-quantum cryptographic system establishing a portable, ephemeral root of trust, comprising: an ingestion interface configured to continuously harvest a time-varying cardiac electrical waveform from a user; and a portable hardware token comprising a physical Trusted Execution Environment (TEE) cryptographic hardware boundary enclosing and isolating: a secure chip enclave executing a continuous liveness monitoring subsystem linked to said ingestion interface, said subsystem configured to extract a dynamic biological feature vector from said waveform and to determine liveness by continuously monitoring a bias voltage associated with said waveform; a room-temperature photonic quantum random number generator (μQRNG) comprising an optical emitter, a waveguide, and a single-photon avalanche diode (SPAD) detector operative at ambient temperature, configured to provide a continuous stream of true quantum entropy derived from quantum-mechanical photon detection events; a hardware cryptographic fusion engine coupled to said secure chip enclave and said μQRNG, said engine configured to deterministically combine said dynamic biological feature vector with said true quantum entropy stream via a key derivation function (KDF) primitive to generate an ephemeral quantum cardiac cryptographic key (KQCK) token; and a hardwired, analog crowbar zeroization circuit, implemented independently of any firmware or software execution, physically connecting said continuous liveness monitoring subsystem to volatile registers of said fusion engine, wherein said circuit is configured to short said registers to a ground plane to flush and erase said ephemeral KQCK token within less than 1.2 microseconds of a detected liveness failure or physical biometric detachment; and an outbound host transport bridge configured to cryptographically wrap said ephemeral KQCK token within a session key or secure channel protocol, and to project the wrapped token, or a proof derived therefrom, outside said physical TEE cryptographic hardware boundary to authenticate user permissions across an external target environment, wherein the unwrapped ephemeral KQCK token is not exposed in the clear during said projection.

2. The system of claim 1, wherein the secure chip enclave includes a hardware fuzzy extractor configured, during an initialization phase, to map statistical autonomic nervous system variations from baseline heart rate variability (HRV) metrics into a stable, reproducible biometric root key, together with helper data, stored in non-volatile memory, that is structurally insufficient on its own to reconstruct the user's underlying biometric signal, wherein said biometric root key is stored as an unexported cryptographic anchor inside an isolated, non-volatile register within said physical TEE cryptographic hardware boundary, said register having no debug, test-mode, or firmware-mediated access pathway.

3. The system of claim 2, wherein said external target environment comprises a secondary, standalone hardware device containing a third-party silicon-bounded trusted execution environment, and wherein said host transport bridge is configured to transmit the wrapped token or derived proof to said third-party trusted execution environment to temporarily augment its security state with a biometric-quantum-derived validation profile, without transmitting the unwrapped ephemeral KQCK token or the biometric root key.

4. The system of claim 1, wherein, in one embodiment, the portable hardware token is physically arranged in an ISO/IEC 7810 ID-1 card form factor powered by an internal lithium polymer (LiPo) battery embedded within said physical TEE cryptographic hardware boundary.

5. The system of claim 1, wherein the host transport bridge is configured to project the wrapped ephemeral KQCK token, or a proof derived therefrom, to authenticate user permissions against an external distributed network or distributed application, while all entropy generation and key derivation are retained entirely within said physical TEE cryptographic hardware boundary.

6. The system of claim 1, wherein said optical emitter comprises gallium arsenide (GaAs) having a zincblende crystal structure, said waveguide comprises silicon dioxide (SiO₂), and said μQRNG is miniaturized using a thin-film lithium niobate (TFLN) integration platform to enable incorporation within a compact wallet-card form factor.

7. The system of claim 1, wherein, in another embodiment, the portable hardware token is physically arranged as a fixed adapter module configured for integration with a legacy access control device or timekeeping device, said adapter module comprising the same physical TEE cryptographic hardware boundary, secure chip enclave, μQRNG, fusion engine, and zeroization circuit recited in claim 1, and further comprising a physical interface configured for fixed or hardwired attachment to said legacy device.

8. The system of claim 1, wherein said continuous liveness monitoring subsystem is configured to detect said liveness failure by detecting a collapse of said bias voltage below a threshold value, said collapse corresponding to a physical detachment of said ingestion interface from said user or a discontinuity in said time-varying cardiac electrical waveform.

9. The system of claim 2, wherein said hardware fuzzy extractor is further configured, during a subsequent authentication cycle, to reconcile a live biological feature vector captured by said ingestion interface against said helper data to recover said biometric root key, without re-exporting or re-exposing the user's underlying cardiac waveform or said biometric root key outside said physical TEE cryptographic hardware boundary.

10. The system of claim 2, wherein the proof derived from said ephemeral KQCK token comprises a zero-knowledge proof, a signed assertion, or a one-time authentication code, wherein said proof does not itself contain recoverable key material corresponding to said ephemeral KQCK token or the biometric root key.

11. The system of claim 1, wherein said host transport bridge communicates externally via a near-field communication (NFC) antenna array, an encrypted Bluetooth Low Energy (BLE) micro-radio, or both.

12. A method for authenticating user permissions to an external system using an ephemeral biometric-quantum root-of-trust credential, comprising: receiving an ephemeral cryptographic key token, said token having been generated from a fusion of a dynamic biological feature vector and a true quantum entropy stream; cryptographically wrapping said ephemeral cryptographic key token within a session key or secure channel protocol prior to transmission via an interface and transport layer; and transmitting the wrapped token, or a proof derived therefrom, to a distributed network, host environment, or third-party secure enclave to authenticate user permissions, independent of any specific physical hardware architecture used to generate said ephemeral cryptographic key token, wherein the unwrapped token is not exposed in the clear during said transmission.

13. The method of claim 12, wherein said external system comprises a host device containing a third-party silicon-bounded trusted execution environment, and wherein transmitting the wrapped token or derived proof to said third-party trusted execution environment temporarily augments its security state with a biometric-quantum-derived validation profile, without transmitting the unwrapped ephemeral cryptographic key token or any permanently-stored biometric root key.

14. The method of claim 12, wherein the proof derived from said ephemeral cryptographic key token comprises a zero-knowledge proof, a signed assertion, or a one-time authentication code, wherein said proof does not itself contain recoverable key material corresponding to said ephemeral cryptographic key token.

15. The method of claim 12, wherein said interface and transport layer comprises a near-field communication (NFC) antenna array, an encrypted Bluetooth Low Energy (BLE) micro-radio, or both.
